(19) United States
(12) Patent Application Publication
(10) Pub. No.: US 2020/0174018 A1
(43) Pub. Date: Jun. 4, 2020

(54) METHOD AND APPARATUS FOR DETERMINING INTERSTITIAL VOLUME

(71) Applicant: PHARMACOPHOTONICS, INC. D/B/A FAST BIOMEDICAL, Carmel, IN (US)

(72) Inventor: Daniel J. Meier, Carmel, IN (US)

(73) Assignee: PHARMACOPHOTONICS, INC. D/B/A FAST BIOMEDICAL, Carmel, IN (US)

(21) Appl. No.: 16/594,693

(22) Filed: Oct. 7, 2019

Related U.S. Application Data
(60) Provisional application No. 62/742,045, filed on Oct. 5, 2018.

(51) Int. Cl. G01N 33/68; G01N 33/70; G16H 10/00; G16H 50/20; G01N 33/58; A61B 5/00; A61B 5/20

(52) U.S. Cl. CPC G01N 33/6893; G01N 33/70; G16H 10/00; G16H 50/20; G01N 33/582; A61K 45/06; A61B 5/201; G01N 2800/347; G01N 2800/52; G01N 2800/56; G01N 2800/50; A61B 5/0071

(57) ABSTRACT

A method and system for selecting a treatment for a subject based on a value for the interstitial space volume of the subject utilizes plurality of sample data values representing concentrations of small and large markers in plurality of blood samples over time. The sample concentrations are utilized to predict a hypothetical peak concentration of the small marker prior to the dissipation of the markers during the test period. This hypothetical peak concentration and other sample values are utilized with either a bi-exponential or tri-exponential decay curve fitting algorithm to define a decay curve, the curve characteristics of which are then utilized to calculate values for glomerular filtration rate, a leakage rate of the small marker into interstitial space, and finally a value for the interstitial volume. The determined value for the interstitial volume can then be compared with number thresholds and decisions made for recommended therapy for the subject, if desired.

### FIELD OF THE INVENTION

The disclosure relates, at least in part, to methods of measurement of biometric indicators in a mammalian subject, and, more particular, to systems and techniques for measuring the volume of the interstitial space as a diagnostic tool for treatment of disease.

### BACKGROUND OF THE INVENTION

Biometric indicators are valuable tools used by medical practitioners to aid in the diagnosis of a patient, and their ability to determine the proper course of medical treatment is often limited by access to rapid and accurate quantitative biometric information. Some common biometric indicators used by medical practitioners include core body temperature, blood pressure, heart and respiratory rates, blood oxygenation and hematocrit, glomerular filtration rate (“GFR”), and the like. While a medical practitioner may prefer to assess multiple biometric indicators prior to deciding on a particular treatment, the patient's condition may deteriorate faster than the indicators may be assessed. In these situations, medical practitioners are required to make decisions with limited information, potentially decreasing a patient's chance of survival. One biometric indicator which could provide a powerful diagnostic tool to medical practitioners is the patient's interstitial volume.

The human body and even its individual body fluids may be conceptually divided into various fluid compartments, which, although not literally anatomic compartments, do represent a real division in terms of how portions of the body's water, solutes, and suspended elements are segregated. The two main fluid compartments are the intracellular and extracellular compartments. The intracellular compartment is the space within the organism's cells and it is separated from the extracellular compartment by cell membranes.

About two thirds of the total body water of humans is held in the cells, mostly in the cytosol, and the remainder is found in the extracellular compartment. The extracellular fluids may be divided into three types: interstitial fluid in the “interstitial compartment” (surrounding tissue cells and bathing them in a solution of nutrients and other chemicals), blood plasma and lymph in the “intravascular compartment” (inside the blood vessels and lymphatic vessels), and small amounts of transcellular fluid such as ocular and cerebrospinal fluids in the “transcellular compartment”. The interstitial and intravascular compartments readily exchange water and solutes but the third extracellular compartment, the transcellular, is thought of as separate from the other two and not in dynamic equilibrium with them. The interstitial compartment (also called “interstitial space”) surrounds tissue cells and is filled with interstitial fluid. Interstitial fluid provides the immediate microenvironment that allows for movement of ions, proteins and nutrients across the cell barrier. This fluid is not static, but is continually being refreshed by the blood capillaries and recollected by lymphatic capillaries. In the average male (70 kg) human body, the interstitial space has approximately 10.5 liters of fluid.

Determination of dry weight of a patient with disease, such as congestive heart failure, hypertension, and chronic kidney disease has always been extremely difficult as there is no commercial and practical way to determine a patient's interstitial volume. Diuretics are used to control total body volume, of which interstitial volume is an important component, by clinician for such diseases. With all of these diseases, quantification of interstitial volume would help to maximize the desired effects of diuretics and minimize the side effects. In addition, understanding the rate of volume increase in the interstitial volume, indicating leakage of fluid from the intravascular compartment, may be used as a measure of endothelial disease/injury in diseases like sepsis, burns, radiation toxicity, edema forming states and some drug toxicities.

Accordingly, a need exists for a system and technique for accurately measuring the interstitial volume.

Accordingly, a need exists for a system and technique for accurately measuring changes in the interstitial volume.

### SUMMARY OF THE INVENTION

The disclosure generally relates to compositions and methods for the measurement of biometric indicators in a mammalian subject. The mammalian subject may be a human. The biometric indicators of interest include, but are not limited to, hematocrit, blood volume, plasma volume, volume of distribution, and glomerular filtration rate (GFR), and interstitial volume. More specifically, a method and system for selecting a treatment for a subject based on a value for the interstitial space volume of the subject utilizes plurality of sample data values representing concentrations of small and large markers in plurality of blood samples over time. The sample concentrations are utilized to predict a hypothetical peak concentration of the small marker prior to the dissipation of the markers during the test period. This hypothetical peak concentration and other sample values are utilized with either a bi-exponential or tri-exponential decay curve fitting algorithm to define a decay curve, the curve characteristics of which are then utilized to calculate values for glomerular filtration rate, a leakage rate of the small marker into interstitial space, and finally a value for the interstitial volume. The determined value for the interstitial volume can then be compared to a number of predetermined thresholds or ranges associated with various morphologies and the determination and/or recommendation can be made as to further treatment for the subject.

Also disclosed are compositions, systems and methods for collecting and analyzing biometric information from a mammalian subject, and more particularly, biometric indicators of interstitial volume, volume of distribution, and glomerular filtration rate.

According to one aspect of the disclosure, a method of selecting a treatment for a subject having or at risk of a disease based on a value for the interstitial space volume of the subject, the method comprising: A) acquiring a plurality of sample data values representing concentrations of a small marker and a large marker in blood samples of a subject over a duration of time, the small marker filterable by glomeruli of the subject and the large marker not filterable by the glomeruli of the subject; B) calculating a value for plasma volume of the subject, V₁, by dividing a dosed concentration of the large marker provided to the subject by a measured average concentration of the large marker from the plurality of sample data values; C) calculating a value for concentration of the small marker at time zero (C₀) using the calculated vale of the plasma volume (V₁); D) fitting the plurality of sample data values of at least the small marker to a curve using the value of C₀; E) calculating a plurality of values for parameters of a resulting fitted curve; F) calculating a value for mGFR using the plurality of values for parameters of the fitted curve and a value for an initial dose of the small marker provided to the subject; G) deriving a value for a measured leakage rate of the small marker into interstitial space of the subject using the calculated value of the mGFR and the calculated plurality of values for parameters of the fitted curve; H) deriving a value for the interstitial space volume of the subject using the derived value for the measured leakage rate of the small marker into the interstitial space and the calculated value of mGFR and the calculated plurality of values for parameters of the fitted curve; and I) selecting one or more treatments for administration to the subject when the derived value for the interstitial space volume exceeds a threshold value for interstitial space volume that would classify the subject as in need of a treatment and/or modulation of treatment for a disease.

According to one aspect of the disclosure, a method of selecting a treatment for a subject having or at risk of developing congestive heart failure, hypertension, chronic kidney disease or sepsis, comprises: A) administering a first VFI to the subject, wherein the first VFI is filtered by the glomeruli of the subject; B) administering a second VFI to the subject, wherein the second VFI is not filtered by the glomeruli of the subject; C) measuring a concentration of both the first VFI and the second VFI in the subject, at a timepoint Tm; D) determining the vascular volume of distribution of both the first VFI and the second VFI at Tm; E) calculating a T₀ concentration (C_{T0}) for the first VFI by one of multiplying the concentration of the second VFI concentration at Tm by the ratio of (first VFI concentration at Tm)/(second VFI concentration at Tm); F) calculating interstitial volume of the subject from the C_{T0} value; and G) if the calculated interstitial volume exceeds a threshold value for interstitial volume that would classify subject as in need of a treatment and/or modulation of treatment, selecting one or more treatments for congestive heart failure, hypertension, chronic kidney disease or sepsis for administration to the subject, thereby selecting a treatment for a subject having or at risk of developing congestive heart failure, hypertension, chronic kidney disease or sepsis. In one embodiment, the method further comprises administering the selected treatment to the subject, optionally via intravenous injection or other techniques.

According to another aspect of the disclosure, a method of selecting a treatment for a subject having or at risk of a disease comprises: A) determining a vascular volume of distribution of both a first VFI and a second VFI at a timepoint Tm, the first VFI filterable by the glomeruli of the subject and the second VFI not filterable by the glomeruli of the subject; B) calculating a T₀ concentration (C_{T0}) value for the first VFI by multiplying a concentration of the second VFI concentration at Tm by a ratio of the first VFI concentration at Tm to the second VFI concentration at Tm; C) calculating interstitial volume of the subject from the C_{T0} value and measured concentrations of the first VFI and the second VFI at Tm; and D) selecting one or more treatments for administration to the subject when a calculated interstitial volume exceeds a threshold value for interstitial volume that would classify subject as in need of a treatment and/or modulation of treatment.

According to another aspect of the disclosure, a method of selecting a treatment for a subject having or at risk of a disease comprises: A) obtaining data representing a measured concentration of both a first VFI and a second VFI in a subject, at a timepoint Tm, the first VFI filterable by the glomeruli of the subject and the second VFI not filterable by the glomeruli of the subject; B) determining a vascular volume of distribution of both the first VFI and the second VFI at Tm; C) calculating a T₀ concentration (C_{T0}) for the first VFI by one of multiplying the concentration of the second VFI concentration at Tm by the ratio of (first VFI concentration at Tm)/(second VFI concentration at Tm), or by a proxy for such comparison of the first VFI concentration at Tm to the second VFI concentration at Tm; D) calculating interstitial volume of the subject from the C_{T0} value; and E) selecting one or more treatments for administration to the subject when a calculated interstitial volume exceeds a threshold value for interstitial volume that would classify subject as in need of a treatment and/or modulation of treatment. In one embodiment, the method further comprises administering the selected treatment to the subject, optionally via intravenous injection or other techniques.

According to yet another aspect of the disclosure, a system for calculating the interstitial volume of a patient comprises: A) a peripheral device operational to measure a concentration of both a first VFI and a second VFI in a subject, at a timepoint Tm, the first VFI filterable by the glomeruli of the subject and the second VFI not filterable by the glomeruli of the subject; B) a memory operational to store a plurality of measured concentration values of the first VF I and second VFI, a plurality of threshold values of interstitial volume and a plurality of treatment recommendations associated with the threshold interstitial volume values; C) a processor, coupled to the peripheral device and memory and operational to: i) determine a vascular volume of distribution of both the first VFI and the second VFI at Tm; ii) calculate a T0 concentration (C_{T0}) for the first VFI by multiplying the concentration of the second VFI concentration at Tm by the ratio of (first VFI concentration at Tm)/(second VFI concentration at Tm), iii) calculate interstitial volume of the subject from the C_{T0} value and measured concentrations at Tm; and D) a presentation device operatively coupled to the processor and the memory and operational to present the calculated interstitial volume. In embodiments, the presentation device is a display device and is further operational to present a user interface enabling selection of one or more treatments for administration to the subject, when the calculated interstitial volume exceeds a threshold value for interstitial volume that would classify subject as in need of a treatment and/or modulation of treatment. In embodiments, the display device is configurable to present any of the calculated interstitial volume, the exceeded threshold value, or any recommended selected treatment to a user. In embodiments, the peripheral device comprises an oral probe adaptable to be placed sublingually within the oral cavity and comprising an optical conduit couple to the processor. In embodiments, the processor comprises spectrometric analyzer.

According to still another aspect of the disclosure, a computer program product for use with a computer system operatively coupled to a peripheral, the computer program product comprising a non-transitory medium having computer readable instructions embedded thereon comprising: A) program code for measuring a concentration of both a first VFI and a second VFI in a subject, at a timepoint Tm, the first VFI filterable by the glomeruli of the subject and the second VFI not filterable by the glomeruli of the subject; B) program code for determining a vascular volume of distribution of both the first VFI and the second VFI at Tm; C) program code for calculating a T₀ concentration (C_{T0}) for the first VFI by multiplying the concentration of the second VFI concentration at Tm by the ratio of (first VFI concentration at Tm)/(second VFI concentration at Tm), or by a proxy for such comparison of the first VFI concentration at Tm to the second VFI concentration at Tm; D) program code for calculating interstitial volume of the subject from the C_{T0} value and measured concentrations at Tm; and E) program code for causing the calculated interstitial volume to be presented on a device. In embodiments, the computer program product further comprises: F) program code enabling selection of one or more treatments for administration to the subject, if the calculated interstitial volume exceeds a threshold value for interstitial volume that would classify subject as in need of a treatment and/or modulation of treatment.

### BRIEF DESCRIPTION OF THE DRAWINGS

Various aspects of the present disclosure are described herein with reference to the drawings in which:

FIG. 1 is an example of the results of a step dose blood test set in accordance with the disclosure;

FIG. 2 is a plot of each VFI component (intercept forced to zero), using the average signal level and amount of each component at each dose step in accordance with the disclosure;

FIG. 3 is a plot of fluorescence intensity level vs. HCT in accordance with the disclosure;

FIG. 4 is plot of the HCT data of FIG. 3 taking the ratio of the signal levels of Component 1 to Component 2, and plotting that ratio versus the HCT calculated at each stage in accordance with the disclosure;

FIG. 5 is an example of a spectrometric data set obtained from administering and fluorescently monitoring of the vascular distribution of an injectate in accordance with the disclosure;

FIG. 6 is an example of a calibration curve of the fluorescence intensity signal level vs. material amount in accordance with the disclosure;

FIG. 7 is an example of a calibration curve of the fluorescence intensity signal level vs. HCT in accordance with the disclosure;

FIG. 8 is an example of a calibration curve of the raw ratio (concentration ratio of the dynamic and static markers at T₀) of the fluorescent markers vs. HCT in accordance with the disclosure;

FIG. 9 is an example of a calibration curve of fluorescent intensity signal level vs. HCT using a single static marker with two fluorescent tags in accordance with the disclosure;

FIG. 10 illustrates a spectrometric data set obtained from a administering and fluorescently monitoring of the vascular distribution of an injectate multiple times over a test period as would be characterized by a normal mGFR in accordance with the disclosure;

FIG. 11 illustrates a spectrometric data set obtained from a administering and fluorescently monitoring of the vascular distribution of an injectate multiple times over a test period as would be characterized by an impaired mGFR in accordance with the disclosure;

FIG. 12 is a graphic illustration a decay rate showing calculated T0 concentration and bi-exponential curve fit of a Patient 23 in accordance with the disclosure;

FIG. 13 illustrates conceptually a prior art two compartment model;

FIG. 14 shows an exemplary screen shot of application of the GFR calculator software in accordance with the disclosure;

FIG. 15 a system, including the GFR calculator application, capable of executing the methods described in accordance with the disclosure;

FIG. 16 is a graphic illustration a decay rate showing calculated T0 concentration and bi-exponential curve fit in accordance with the disclosure;

FIG. 17 is a graphic illustration a decay rate showing calculated T0 concentration and three-exponential curve fit in accordance with the disclosure; and

FIG. 18 is a flowchart of the process for calculating the interstitial volume of the subject based on a plurality of marker concentration samples in accordance with the disclosure.

### DETAILED DESCRIPTION OF THE INVENTION

For the purposes of promoting an understanding of the principles of the disclosure, reference will be made to the embodiments illustrated in the drawings and specific language will be used to describe the same. It will nevertheless be understood that no limitation of the scope of the disclosure, with such alterations and further modifications in the illustrated system and method and such further applications of the principles of the technology as illustrated therein being contemplated as would normally occur to one skilled in the art to which the technology relates.

As defined in the present application, the term “plasma volume” refers to the total amount of plasma contained in the vasculature of a subject, while the term “circulating plasma volume” refers to the amount of flowing plasma contained in the vasculature of the subject. Although the measurements for “plasma volume” and “circulating plasma volume” are similar and related, they are not the same.

Biometric indicators such as hematocrit, glomerular filtration rate and plasma volume may be measured by administering an injectate with a dynamic fluorescent marker (i.e., a dynamic molecule labeled with a first fluorescent tag) and a static fluorescent marker (i.e., a static molecule labeled with a second fluorescent tag, wherein the first and second tags have distinct (non-overlapping) fluorescent characteristics that enables them to be separately detected) into the vasculature of the mammalian subject. Biometric indicators such as hematocrit and plasma volume (but not GFR) may also be determined by administering an injectate containing a single static marker labeled with two fluorescent tags, into the vascular system of the subject. The markers of the disclosure may also be described herein in terms of a fluorescent tag being “conjugated” to or “associated with” a static or dynamic marker. This terminology is not meant to imply any particular chemical means by which the dynamic or static molecule is “labeled” with the tag. The methods entail measuring the emission intensities of the fluorescent tags over a period of time with one or more measurements, depending on the indicator that is being determined. For example, PV may be measured via a single measurement (as the term is used herein) whereas GFR may be determined on the basis of three measurements conducted at predetermined times after administration of the injectate.

The methods of the disclosure may be practiced with an injectate, also referred to herein as an “visible fluorescent injectable (VFI)”, a device or group of devices capable of measuring fluorescent intensity, and a group of mathematical algorithms capable of determining different biometric indicators based upon the data collected from the VFI and the device(s). As disclosed herein, the VFI may, in some embodiments, include two dextran molecules, of differing molecular weights, conjugated to 2 fluorescently distinct tags, e.g., dyes. Thus, in some embodiments, a first high molecular weight dextran molecule may be conjugated to a fluorescent “red dye”, and another, low molecular weight dextran may be conjugated with fluorescent “green dye.” The “device” may be a probe-based instrument such as a ratiometric fluorescent device (RFD), which is designed to work in concert with a probe, such as an invasive probe, e.g., one designed to insert into the vein of a mammalian subject, as well as with non-invasive probes, e.g., oral probes that are capable of measuring fluorescent intensity through the skin of the mouth. Other devices that may be used in the practice of the disclosure include blood sample reading devices, such as clinical lab-based instruments that use blood samples spun down to yield plasma, and bedside instruments that are capable of reading fluorescence through whole blood, in accordance with the present invention, require a “correction” in order for accurate determination of HCT. Each biometric indicator that can be measured in accordance with the disclosure requires different parts of the data set collected by the devices, in different mathematical equations used to obtain the necessary measurements. Illustrations of such equations and measurements are illustrated in the working examples.

Plasma volume (PV) may be determined using a single static marker which has two fluorescently distinct tags conjugated thereto. PV may be derived by optionally taking a blank (pre-dose) sample to measure residual or background or existing fluorescence, followed by a measurement of fluorescent intensity of the tags after “distribution” of the marker occurs, e.g., usually in about 10-15 minutes following administration of the injectate. As used herein, the term distribution refers to a time when the marker (or markers) has mixed thoroughly into the blood plasma. The data set is then used to calculate PV by measuring concentration of the large marker in the blood plasma (VFI (dose concentration) ones divided by the measured concentration). This value directly measures PV. Optionally, additional samples can be taken over time to monitor changes in PV. This monitoring allows a clinician to perform interventions and thus monitor how PV has changed. In turn, blood volume can be derived by adding back to the total volume, the amount of HCT contained in the subject. The HCT total volume plus plasma volume is equal to blood volume.

Glomerular Filtration Rate (GFR) can also be determined in several ways using the present disclosed methods. In some embodiments, blood samples are taken at different time points. Persons skilled in the art may optionally take a blank (pre-dose value) measurement, which is used to determine any residual/background/existing level of fluorescence. This measurement is especially advantageous in those embodiments in which repeat or follow-on dosing of the VFI is conducted. Data are then collected from samples at about 3 time points, e.g., 10-15 minutes, about 60 minutes and about 120 minutes. A calculation at T₀, which is done by using the PV value of the large marker and dividing by the concentration of the small marker in the VFI, enables persons skilled in the art to derive the rapid phase of the clearance between T₀ and the time point at 10-15 minutes. Once equilibrium has been established (as the term is used herein), the data sets obtained from the measurements at about 60 and at about 120 minutes allows for the determination of the slow phase clearance. Then, one may mathematically derive an area under the curve (AUC) of the total clearance, yielding the GFR. Alternatively, GFR may be determined using a probe-based system, which may entail first generating an HCT value (as described herein) and then collecting data sets before the VFI is injected (pre-dose), another data set prior to equilibrium (e.g., before and up to about 10-15 minutes), and then at post-equilibrium (about 60-120 minutes). These data sets are then used in the same way as described above in the context of blood samples. Whole blood samples can be used without spinning down to isolate plasma, but in these embodiments, persons skilled in the art would need to know the HCT value (which can be measured in accordance with standard techniques, such as by capillary centrifuge). Subsequent data sets can be taken, e.g., at 120 and 180 minutes, etc., and which can be used to update the value of the slow clearance phase, and a new AUC curve derived to show changes in GFR over time.

Hematocrit (HCT) may be used for correction of the probe-based system with a RFD device. These devices are capable of continuous readings of a fluorescent signal in whole blood that is flowing within the body. The disclosure utilizes data sets taken at certain times to produce the HCT. Thus, in the case of probes, a data set taken within the first 10-15 minutes can be used to extrapolate back to the intensity that would have been determined at T₀ (time 0, which as used herein, refers to a point that cannot be measured directly but can be mathematically derived by curve fitting equations to yield an intensity equivalent to the starting concentration of the fluorescent tags in the VFI). The raw ratio of the two intensities (e.g., green versus red tags or dyes) can then be used to determine the HCT value, which can be done by using a previously-calibrated HCT curve of the mammalian subject being tested.

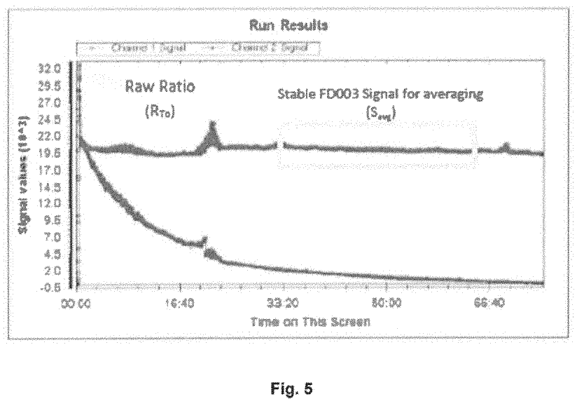

Hematocrit may be determined by analyzing a spectrometric data set, as shown in FIG. 5, obtained from the administration and fluorescent monitoring of the vascular distribution of an injectate for a period of time that includes the peak vascular distribution of the markers at T₀. A calibrated spectrometric analyzer may be used to determine HCT from the spectrometric data set. One advantage of an aspect of the disclosure is the ability to utilize dynamic and static markers to determine HCT in a subject.

A spectrometric data set as used in the present application means a data set resulting from the administration and fluorescent monitoring of the vascular distribution of an injectate containing two or more fluorescent markers of distinct fluorescent characteristics, where one of the fluorescent markers is a dynamic marker and one of the fluorescent markers is a static marker, or wherein both fluorescent markers are associated with a static molecule, for a period of time that includes the peak vascular distribution of the fluorescent markers.

A calibrated spectrometric analyzer useful with the disclosed techniques includes an input for a spectrometric data set, an input for calibration identification, a computational engine for calculating hematocrit, and an output for reporting a calculated hematocrit. The calibration identification may be set with factory predicted average injectate parameters during manufacturing and stored in a computationally accessible location, it may be updated indirectly via a change in software or hardware, or may be updated directly by uploading injectate specific parameters. Injectate specific parameters may be inputted through the use of a manual device, such as a keypad or touch screen, through the use of a semi-automated device, such as a barcode scanner, or through the use of an indirect automated process, such as by the use of a wireless software update.

### Algorithms and Application

An algorithm was used to determine the mGFR and PV from the measured plasma concentrations of the two markers. The critical starting point for the decay curve, T0, is impossible to measure directly, as the VFI immediately begins the process of being distributed throughout the body once injected, but can be derived if the vascular volume is known in accordance with the techniques disclosed herein. The vascular volume of distribution can be determined by measuring the plasma concentration of the non-filtered marker using the following equation:

$${V}_{1} = \frac{D}{{C}_{1}}$$

Where V₁ is the plasma volume, D is the dose in mg of the non-filtered large marker, and C₁ represents the measured concentration of the large marker in the plasma.

The VFI used for the mGFR measurement contains a known concentration of both filtered and non-filtered markers, which can be used to derive the T0 concentration by multiplying the concentration of the large, non-filtered marker by the ratio of the concentrations. For example, assume the VFI contains 35 mg/ml of filtered marker and 15 mg/ml of un-filtered marker; a concentration ratio of 2.33. By multiplying the concentration of the un-filtered marker determined between 10 and 15 minutes by 2.33, the concentration of the filtered marker at T0 can be determined. The ability to properly calculate this starting concentration is unique to the dual markers of the mGFR test of the instant disclosure. The concentration of the filtered small marker at T0 can then be used as a starting point for a decay curve fitting calculation.

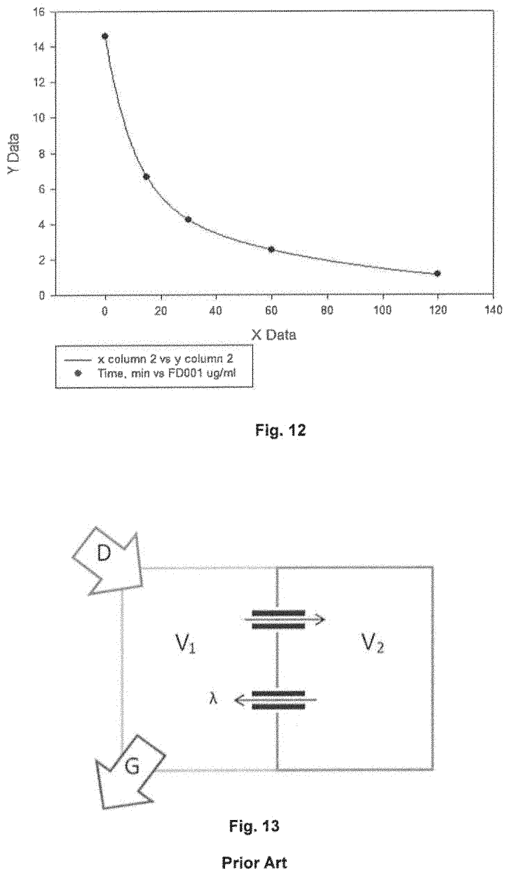

Results from the Phase 1 human trial have shown that the concentration decay of the small filtered marker is very rapid during the first 15 minutes. In healthy patients more than half the starting concentration has decayed in that time, as shown in FIG. 12. FIG. 12 demonstrates a decay rate showing calculated T0 concentration and bi-exponential curve fit of Patient 23.

This rapid decay makes it almost impossible to properly predict the T0 point using only the curve fit of later sample times. Current single marker GFR tests rely on much longer test times to compensate for missing this early data, but in many cases large errors, as high as 30%, can still be present unless the slow curve is followed out to 8 hours or more.

Sapirstein, L. A., Vidt, D. G., Mandel, M. J., & Hanusek, G. (1955), Volumes of Distribution and Clearances of Intravenously Injected Creatinine in the Dog, American Journal of Physiology-Legacy Content, 181(2), 330-336, describe the derivation of modeling a two compartment system and show that two critical boundary conditions exist for the bi-exponential decay of C₁. The following equations use the two compartment model shown in FIG. 13. The dose “D” is introduced into volume V₁. There is a constant exchange, resulting in mixing and distribution of the dose, between volumes V₁ and V₂ at a rate of λ while the kidneys are continuously pulling from volume V₁ at rate G (the GFR).

Using the following Boundary Conditions:

$${C}_{1} = {Ae}^{- α t} + {Be}^{- β t}$$

$$at t = 0 , {C}_{0} = \frac{D}{{V}_{1}}$$

$$at t = 0 , {C}_{2} = 0$$

### Therefore:

$$\frac{D}{{V}_{1}} = A + B = {C}_{0}$$

$${V}_{1} = \frac{D}{A + B}$$

where C₁ is the concentration of the filtered marker over time, t, in minutes, C₀ represent the concentration of the vascular space, V₁, at t=0, and C₂ represent the concentration within the interstitial space, V₂, at t=0.

When using the herein described dual marker algorithm, the above-noted boundary conditions are valid, as the equations derived from the boundary conditions shown above are tested after the curve fit is completed. However, most single marker systems in the art will show a falsely large volume for V₁.

Utilizing the system and algorithms disclosed herein, the measured concentrations are fit to a bi-exponential decay curve with a modified four parameter exponential decay using the mGFR computer program module 209, as described herein. This program module uses the Levenberg-Marquardt method of non-linear least squares. All data are assumed to have homoscedasticity over the two hour interval of the test.

This curve fit yields the following parameter information:

α=slope of steep line

β=slope of shallow line

A=intercept of steep curve

B=intercept of shallow curve

Based on the following equation below:

C₁=Ae^{-αt}+Be^{-βt}

Once the above curve fit parameters are obtained, the below equations are used to calculate the following results:

G=GFR in mL/min

λ=vasculature leakage

V₁=first volume of distribution (plasma)

V₂=second volume of distribution (interstitial volume)

### Glomerular Filtration Rate

$$G = \frac{D α β}{A β + B α} = \frac{D}{\frac{A}{α} + \frac{B}{β}}$$

### Volumes of Distribution

$${V}_{1} = \frac{D}{A + B}$$

$${V}_{2} = \frac{λ G}{{V}_{1} αβ}$$

### Vasculature Leakage

$$λ = \frac{{V}_{1} ( A α + B β )}{A + B} - G$$

### Injectates

In one embodiment, the injectate of the disclosure includes a first fluorescent marker, a second fluorescent marker, and an injectate carrier. Each fluorescent marker has its own distinct fluorescent characteristics, i.e. distinct excitation wavelengths and emission wavelengths. The first fluorescent marker has a first excitation wavelength and a first emission wavelength. The second fluorescent marker has a second excitation wavelength and a second emission wavelength. A fluorescent marker is any molecule containing a fluorophore (also defined to herein as a tag such as a dye) which causes the molecule to be fluorescent. Many known fluorescent dyes can serve as fluorescent markers with the disclosed technique, such as but not limited to rhodamine dyes or its derivatives (e.g., 2-sulfhydroRhodamine (2SHR) and Texas Red®), fluorescein or its derivatives (e.g. fluorescein isothiocyante (FITC)), coumarin and cyanine, all of which have distinct excitation and emission wavelengths from each other. The fluorescent tag may be associated with, for example via conjugation, another macromolecule (a labeled macromolecule) to provide an intended molecular weight for the fluorescent dye. Examples of macromolecules include, but are not limited to, polymers, proteins, dextrans, celluloses, carbohydrates and nucleic acids. The macromolecules can be naturally occurring compounds, or synthetic compounds. Methods for conjugating macromolecules with fluorescent dyes are well known in the art.

The first fluorescent marker is a dynamic molecule labeled with a first fluorescent tag, and the second fluorescent marker is a static molecule labeled with a second fluorescent tag.

A “dynamic molecule” is a molecule of sufficiently low molecular mass to permeate the blood vessel walls or the vasculature of a subject. Dynamic molecules are known in the art to have a molecular mass less than 50 kDa, and more typically have a molecular mass less than 20 kDa.

A “static molecule” is a molecule of sufficiently high molecular mass to significantly limit its blood vessel wall permeability. Static markers may reach a quasi-stable vascular concentration for a period of time, although such markers may ultimately be cleared from the vasculature. Static markers are known in the art to have a molecular mass greater than 50 kDa, and more typically have a molecular mass greater than 200 kDa. Such markers can remain in the vasculature for a time period of between about 1 or 2 hours, to 12 hours or longer, depending on the molecular mass of the marker as well as other factors.

Thus, by way of example, a first fluorescent marker may include a dynamic molecule such as a 5-7 kDa dextran, conjugated to a fluorescein dye, and the second fluorescent marker may include a static molecule such as a 150 kDa dextran conjugated to 2SHR.

In another embodiment, the injectate may include a static marker having two fluorescent tags attached thereto and an injectate carrier. Each fluorescent tag has its own distinct fluorescent characteristics, i.e. distinct excitation wavelengths and emission wavelengths. An example of such a static marker is a macromolecule, such as dextran with molecular mass greater than 50 kDa, labeled with (e.g., conjugated with) two different fluorescent dyes, such as Texas Red® and fluorescein or a derivative thereof.

The fluorescent markers are not metabolized within the subject during the period of time of measuring the biometric indicators. A marker is “not metabolized within the subject” in the present disclosure if the marker has a half-life (T_{1/2}) of approximately 4 hours or greater in the vascular system of the subject.

In the present disclosure, the two injectates can be used substantially interchangeably. That is, with the exception of measuring GFR, it is not important whether the injectate has two separate fluorescent markers providing two distinct fluorescent characteristics, or whether the injectate has only one marker having two fluorescent tags providing two distinct fluorescent characteristics. What is important is that the injectate provides two distinct fluorescent emission signals in order to allow the measurement of the biometric indicators as described in the present application. Thus, when reference is made to using an injectate having two fluorescent markers in the present application, this is also intended to include and refer to an injectate having only one marker but with two fluorescent tags on the molecule. The subsequent steps leading to the measuring of the hematocrit and other biometric indicators are otherwise identical. However, since the injectate including only one molecule uses a static marker without a dynamic marker, the injectate can be used to measure the hematocrit and other biometric indicators, but not GFR which requires at least two markers.

The term “injectate carrier” as used in the present application means a biologically acceptable fluid capable of solubilizing and delivering the fluorescent markers to aid in the delivery and biocompatibility of the fluorescent markers. Examples of suitable carriers include but are not limited to buffers, saline (e.g., physiologically buffered saline) and the like.

The injectate may be introduced into the vascular system via bolus injection or by infusion.

### Calibration Identification and Calibration Identifier

The injectate of the disclosure is calibrated to provide a calibration identification that contains parameters of the injectate.

The term “calibration identification” as used in the disclosure means a collection of fluorescent injectate parameters that are used in the calculation of the biometric parameter such as HCT from a spectrometric data set. The parameters may include the Visible Fluorescence Injectate (VFI) lot number and calibrated fluorescent intensity of each fluorescent marker or each fluorescent tag on the same marker.

A calibration identification can be represented as a calibration identifier represented by a series of numbers or signals. In an embodiment, the series of numbers or signals may be an optical machine-readable representation of data, such as but not limited to bar codes. Algorithms to convert the calibration identifier to a bar code calibration identifier are well known to those in the art.

The calibration identification may be set with factory predicted average injectate parameters during manufacturing, and is stored in a computationally accessible location. It may be updated indirectly via a change in software or hardware, or may be updated directly by uploading injectate specific parameters.

Injectate-specific parameters contained in the calibration identification may be inputted into another device, such as a fluorescent detector or a spectrometric analyzer, through the use of a manual device, such as a keypad or touch screen, through the use of a semi-automated device, such as a barcode scanner, or through the use of an indirect automated process, such as a wireless software update.

The reference standard fluorescent intensities used to generate the calibration curves which in turn are used to calculate the biometric parameters may be represented in the calibration identifier as set value 1000 with an immediately following letter designation for each fluorescent marker of different fluorescent wavelength immediately following (i.e., 1000a; 1000b). Fluorescent intensity variance from the reference standard for each fluorescent marker may be represented in the calibration identifier as a representative equivalent increase or decrease to the set value of 1000.

A sample calibration identifier is shown below:
LOTIOIAI034B0975

which contains the following information:
VFI Lot No.: 101
Fluorescent Marker 1 (A) intensity from calibration: 1034
Fluorescent Marker 2 (B) intensity from calibration: 0975

### Calibrated Injectate

A calibrated injectate of the disclosure (“Calibrated Injectate”) may include a first fluorescent marker or fluorescent tag having a first hematocrit-dependent fluorescent attenuation coefficient, a second fluorescent marker or fluorescent tag having a second hematocrit-dependent fluorescent attenuation coefficient, an injectate carrier, and a calibration identification. The calibration identification may be provided separately from the Calibrated Injectate, may be provided with the Calibrated Injectate, or may be provided as a Calibration Identification. A Calibrated Injectate may be used to further improve the accuracy and precision of a calibrated spectrometric analyzer by correcting for the optical batch variance resulting from the multiple manufacturing steps.

A calibration method of the disclosure used to produce a Calibrated Injectate may include a set of fluorescent intensity standards for each fluorescent marker or fluorescent tag, a set preparation procedure for creating working standard solutions and calibration solution for calibrating a fluorescence detector, and a fluorescence detector used to read the fluorescent intensity of each fluorescent marker in calibrations solution and injectate. From fluorescent marker standard solutions the set procedure is followed to create a working standard solution and a calibration solution. The calibration solution is used in the same fluorescent intensity range for each marker as the injectate. The calibration solution is used to set the parameters of the fluorescence detector. Then using the same set procedure, a test solution is made using the injectate to be calibrated. Using the calibrated fluorescence detector, the injectate test solution for the calibration identification for a Calibrated Injectate is generated.

### Method for Determining Hematocrit

Hematocrit may be determined by analyzing a spectrometric data set obtained from administering and fluorescently monitoring of the vascular distribution of an injectate containing two or more fluorescent markers of different fluorescent wavelengths, where at least one of the fluorescent markers is a dynamic marker, for a period of time that includes the peak vascular distribution of the markers. Alternatively, the injectate may contain only one static marker having two fluorescent tags on the marker. A calibrated spectrometric analyzer may be used to determine HCT from a spectrometric data set. An advantage of the disclosed method and system is its ability to utilize a combination of dynamic static markers, or a static marker (associated with two distinct fluorescent tags) to determine HCT in an animal subject.

The term “time zero” or “T₀”, as used herein, is the point in time that the injectate is introduced into the vasculature of the mammalian subject. It may also coincide with the moment in a spectrometric data set that is characterized by the peak fluorescent signal intensity of intravenously injected fluorescent markers (and thus the point of initial analysis for the mathematical computations). Thus, T₀ is used to signify the start of the biometric parameter fluorescent signal analysis. The term “raw ratio” as used herein may be defined as the ratio of fluorescent signal intensities of the two fluorescent tags at T₀, i.e. the ratio of the dynamic marker (“small marker”, indicating a smaller molecular weight, or “green marker”, indicating a fluorescent tag emitting in the green spectrum) to the static marker (“larger marker”, indicating a larger molecular weight, or a “red marker”, indicating a fluorescent tag emitting in the red spectrum). An important aspect of the present technology is the use of the raw ratio to determine HCT in an optically dynamic environment.

It has been found that up to one-half of the small marker is filtered from the blood stream after only about 15 minutes following the initial bolus infusion of a dynamic marker and a static marker, which in embodiments may total about 3 ml. Accordingly, following the procedure of the present disclosure, the concentration of the dynamic marker at T₀ can be accurately predicted using, for instance, a spectrometric analyzer to measure the concentration of the static marker at 10 to 15 minute intervals as described herein. This is a significant advance since it permits the use of periodic biometric sampling, e.g., sampling the vasculature every 10 to 60 minutes or 3 times over 2 hours (which may be done to calculate plasma volume and GFR), as contrasted to a continuous sampling procedure. Thus the total test time can be shortened to about 1 to 2 hours in duration from about 6 hours required by the current methods. The “sampling” may be conducted in accordance with techniques known in the art, e.g., via blood samples and use of invasive (e.g., venous) or non-invasive (e.g., oral) probes.

The raw ratio may be used, in turn, to calculate the hematocrit observed at the optical interface of an optical probe, referred to herein as the apparent HCT. The apparent hematocrit obtained from invasive (e.g., venous) probes may be different from a subject's true HCT. This may be attributed to fluid dynamic anomalies occurring near an optical interface inserted in a flowing system. True HCT may be calculated from apparent HCT by applying a correction factor. A correction factor may be in the range of 1 to 10 percent of apparent HCT, and more specifically in the range of 4 to 5 percent of HCT. A typical calculation of the correction factor is shown in the Examples herein. Thus, a correction function is not necessary when the disclosed method is carried out with non-invasive probes such as oral probes.

A method for determining a species specific HCT curve may utilize the following components: a calibrated fluorescence detector, a Calibrated Injectate, and a test volume of species specific blood. A procedure may be performed to maintain a constant total test volume and constant concentration of Calibrated Injectate in the test volume while altering the HCT in the test volume. A calibrated fluorescence detector is set up and configured to read the fluorescence intensities of the test volume throughout the procedure. A test volume is prepared, with a known HCT (H_{calib}), as determined by conventional methods, and a measured total volume (Vₜ). A known volume of Calibrated Injectate is added to the test volume. A separate volume is created from normal saline and Calibrated Injectate, with an equivalent concentration of Calibrated Injectate added to the test volume. This solution is used to replace removed volume from the test volume during the procedure. A series of repetitive steps is then used to create different HCT levels in the test volume. A volume (x) is removed from the test volume, discarded, and replaced with an equivalent volume (x) of prepared saline solution. The system is allowed to stabilize, and the HCT is calculated at each stage based on the dilution of HCT. The average signal level of a “flat portion” of data at each HCT level tested is determined as shown, where Vₜ is the total volume in the test set, Vₑ is the volume exchanged (blood for saline), H⁰ is the starting HCT (prior to volume exchange) and H′ is the new HCT (post volume exchange). A hematocrit dependent curve is produced where the raw ratio is an input and the apparent hematocrit is the output.

Invasive, e.g., venous probes suitable for use in the disclosure are known in the art. See, e.g., U.S. Patent Publication 2012/197136, commonly owned, contents of which are hereby incorporated by reference.

### EXAMPLES

Table 1 contains a summary of definitions of the variables used in the following Examples.

TABLE 1

| Variable | Unit | Description |
|---|---|---|
| x₁; x₂ | mg | mg of VFI component 1; component 2 |
| Vₜ | mL | Total blood volume |
| V_{S} | mL | Volume of saline used for HCT calibration curve |
| | | generation |
| V_{D} | mL | Volume of VFI dose given |
| H_{calib} | % | Percentage (decimal) of hematocrit known for whole |
| | | blood used in calibration tests |
| D₁; D₂ | mg/mL | Concentration of component 1 or component 2 of |
| | | VFI given in a dose |
| S₁; S₂ | — | Signal level in generated calibration for |
| | | Component 1; Component 2 |
| m₁; m₂ | mg⁻¹ | Slope of calibration curve for component 1; |
| | | component 2 |
| Vₑ | mL | Volume of blood-saline exchanged in hematocrit |
| | | calibration |
| H^{o} | % | Starting hematocrit prior to each volume exchange |
| H′ | % | Ending hematocrit after each volume exchange |
| S₃; S₄ | — | Signal level in generated in HCT calibration for |
| | | Component 1; Component 2 |
| m₃; m₄ | — | Slope of Component 1 HCT calibration curve; |
| | | Component 2 |
| r | — | Rate of attenuation of component 1 |
| H | % | Hematocrit of the relevant sample |
| b | — | Constant intercept in component 2 hematocrit |
| | | calibration curve |
| R | — | Raw ratio calculated from the hematocrit calibration |
| | | curves for component 1 and 2 |
| K | — | constant of the Ratio vs HCT calibration curve |
| q | — | Rate of attenuation of the ratio of component 1 and 2 |
| R_{To} | — | Ratio of component 1 and component 2 signal levels |
| | | at time zero; from a test |
| S_{avg} | — | Average stable component 2 signal calculated from |
| | | a test |
| Hₐₚₚ | % | Apparent hematocrit calculated from the raw ratio |
| S_{calib} | — | Signal level calculated from calibration curve and |
| | | H_{calib} |
| Sₐₚₚ | — | Signal level calculated from calibration curve and |
| | | Hₐₚₚ |
| C | — | Correction factor applied to S_{avg} to account for |
| | | hematocrit difference |
| S_{C} | — | Signal level after correction factor has been applied |
| x_{eq} | mg | Calculated equivalent amount of material in a test to |
| | | amount used in calibration |
| V_{dist}_{_{calib}} | mL | Volume of distribution of calibration tests |
| x_{sub} | mg | Amount of a VFI component given to a test subject |
| V_{dist}_{_{sub}} | mL | Calculated volume of distribution of the test subject |
| H_{sub} | % | Test subject's hematocrit calculated from the apparent |
| | | HCT and a constant offset |
| Hₒₛ | % | Constant offset between H_{sub} and Hₐₚₚ caused by |
| | | fluid dynamics in the system |
| BV | mL | Blood volume of the test subject |

### Example 1: Method for Generation of Calibration Curves

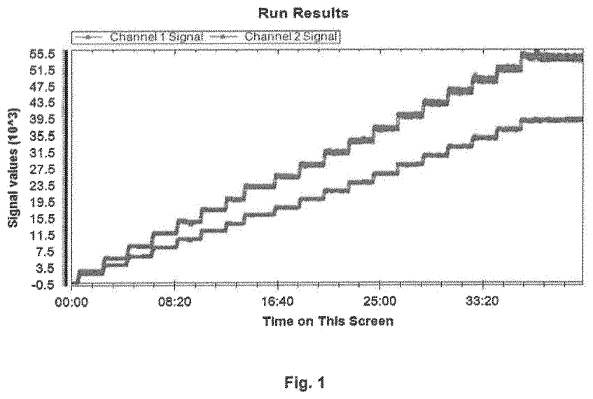

1. A step dose blood test set is run on a whole blood sample containing two fluorescent markers each having its distinct emission wavelength. An example of the results is shown in FIG. 1 with the upper curve representing the first emission signals from the first fluorescent marker or tag recorded in Channel 1 as the Channel 1 signal, and the second emission signals from the second fluorescent marker or tag recorded in Channel 2 as the Channel 2 signal. As discussed previously, this step dose blood test set can also be generated using one static marker having two fluorescent tags each tag having its distinct emission wavelength. Each fluorescent marker or each fluorescent tag may be referred to as a “fluorescent component” hereafter.

2. The average signal level of the “flat” or stable portion at each dose step for each fluorescent component is calculated.

3. Based on the known volume of blood (Vₜ) used, the known dose of VFI (V_{D}) and the known concentration of each VFI fluorescent component (D₁ or D₂) in the dose; the amount of each fluorescent marker present in the blood is calculated at each dose step (1).

x_{1;2}=V_{D}[D_{1;2}] (1)

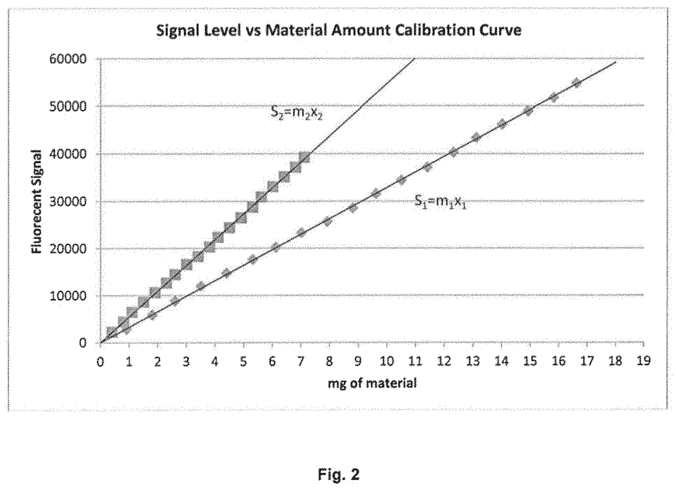

4. A fit line for the plot of each fluorescent component (intercept forced to zero) is generated, using the average signal level and amount of each component at each dose step calculated previously. The plot is shown in FIG. 2.

S₁=m₁x₁ (2)

S₂=m₂x₂ (3)

Where S is the signal level, m is the slope of the fit line and x is the amount (mg) of the material.

### Example 2: Method for Generation of a Species Specific Hematocrit (HCT) Calibration Curve

1. A blood test is run with the single dose approach. With a known volume of blood (Vₜ) and a known HCT of the blood (H_{calib}), the volume of saline (V_{S}) needed for the test is calculated.

Vₜ−VₜH_{calib}=V_{S} (4)

2. The blood and the saline are equivalently dosed from the same VFI vial.

3. A predetermined volume of blood is removed from the test set and discarded. The same volume of dosed saline, as the blood previously removed, is injected back into the test set. This exchange will maintain the concentration of each component as well as the total volume of the test set, but alter the volume of distribution to HCT ratio. This step is repeated numerous times to generate multiple data points at which the volume of distribution and HCT ratio are different.

4. Each new point is allowed to stabilize before a new point is generated. A new HCT is calculated at each stable point.

$$\frac{( {V}_{t} - {V}_{e} ) ( {H}^{0} )}{{V}_{t}} = {H}^{′} \qquad ( 5 )$$

Where Vₜ is the total volume in the apparatus, Vₑ is the volume exchanged (blood for saline), H⁰ is the starting HCT (prior to volume exchange), and H′ is the new HCT (post volume exchange).

5. The average signal level of a “flat” stable portion of data is taken at each HCT level generated during the test.

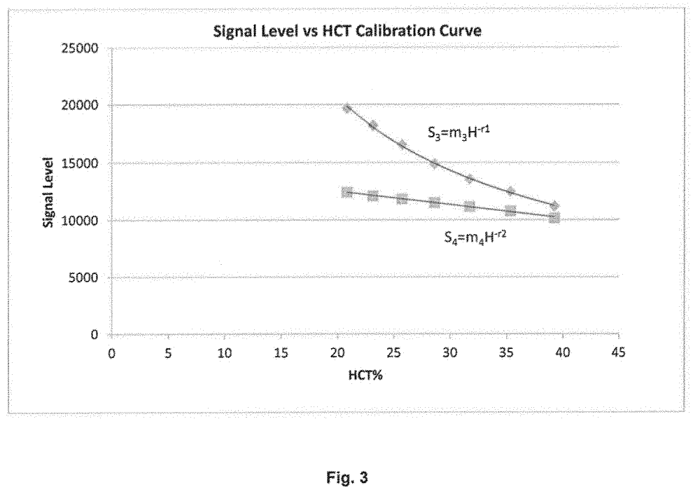

6. A plot of signal level vs. HCT is generated using the values calculated previously, as shown in FIG. 3.

7. A fit line is generated for each of the individual component plots. The equations generated are in the form:

S₃=m₃H^{-r1} (6)

S₄=m₄H^{-r2} (7)

Where S is the signal level, H is the HCT, m is the slope, and r is a rate.

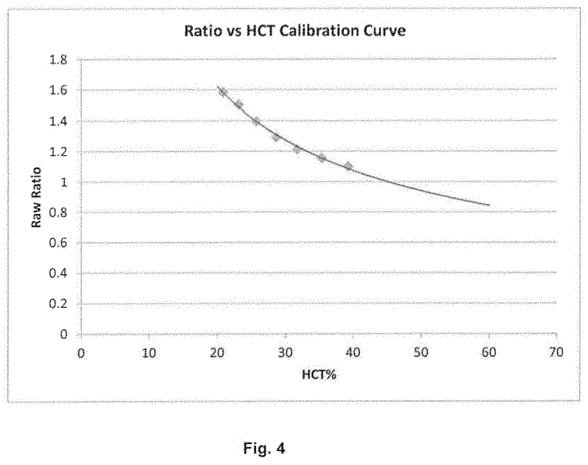

8. A fit line from the same HCT data taking the ratio of the signal levels of Component 1 to Component 2 (8), and plotting that ratio versus the HCT calculated at each stage in equation 5 is generated, as shown in FIG. 4.

R=S₃/S₄ (8)

The equation generated should take the form:

R=KH^{-q} (9)

Where R is the ratio, K is a slope, H is the HCT and q is a rate.

### Example 3: Method for Determining Various Biometric Indicators

When a test is run on a subject, the “batch” of VFI must be known because the signal calibration and HCT calibration curves used for interpretation must be based on the same “batch” of VFI given to the subject.

1. From a test data sample of FIG. 5, the raw ratio at T₀ (R_{T0}) and the average stable Component 2 (FD003) signal level (S_{avg}) are extracted. The lower curve in FIG. 5 represents Channel 1 signals, and the upper curve represents Channel 2 signals.

2. Using the raw ratio at T₀ (R_{T0}), the apparent HCT of the subject is calculated from the Ratio vs HCT Calibration Curve.

R_{T0}=KH_{-q} (10)

H=Hₐₚₚ (11)

3. Using the calculated apparent HCT and the Signal Level vs. Material Amount Calibration Curve; the amount of correction, C, is calculated and applied to the average signal level component.

From Equation 7:

S_{calib}=m₄H_{calib}^{-r} (12)

Sₐₚₚ=m₄Hₐₚₚ^{-r} (13)

If Hₐₚₚ<H_{calib} then S_{calib}/Sₐₚₚ

If Hₐₚₚ>H_{calib} then Sₐₚₚ/S_{calib}

S_{calib}/Sₐₚₚ=C (14)

4. The correction factor, C, calculated in (14), is applied to the average signal level of component 2, S_{avg}, from the test data.

C*S_{avg}=S_{C} (15)

5. The corrected signal, Sc, is used in equation (16) to determine the equivalent amount of material of component 2 based on the Signal Level vs. Material Amount Calibration Curve.

S_{C}=m₂x (16)

x=x_{eq} (17)

6. From a ratio of the known amount (mg) of VFI component 2 dosed in the subject, x_{sub}, to a known volume used in calibration, V_{distcalib}, and a calculated equivalent amount of component 2 (mg), x_{eq}, to a volume of subject's V_{distcalib}, the subject's volume of distribution is calculated.

V_{distcalib}=Vₜ−VₜH_{calib} (18)

x_{eq}/V_{distcalib}=x_{sub}/V_{distsub} (19)

V_{distsub}=x_{sub}V_{distcalib}/x_{eq} (20)

7. The subject's HCT from the apparent HCT and the HCT offset are calculated.

H_{sub}=Hₐₚₚ+Hₒₛ (21)

8. The blood volume from the volume of distribution of the subject and the calculated subject HCT is calculated.

BV=V_{distsub}/H_{sub} (22)

### Example 4: Example Calculation

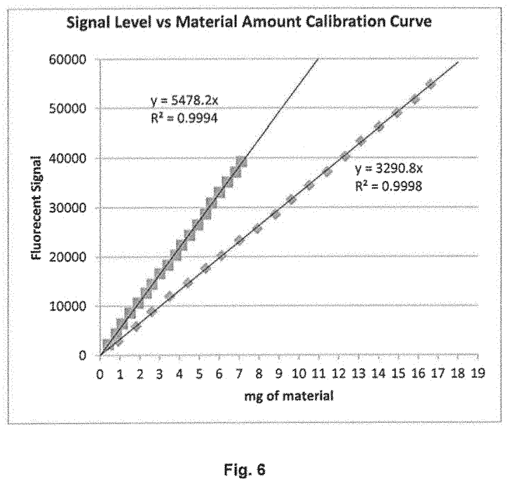

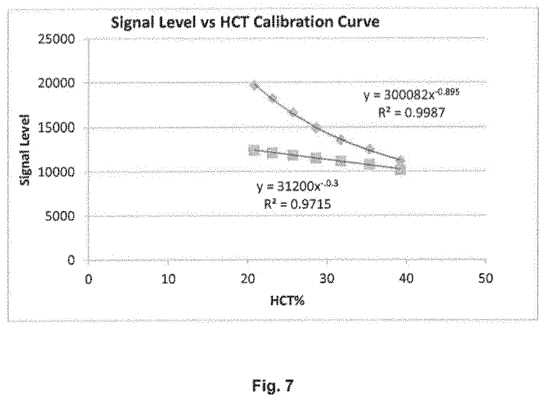

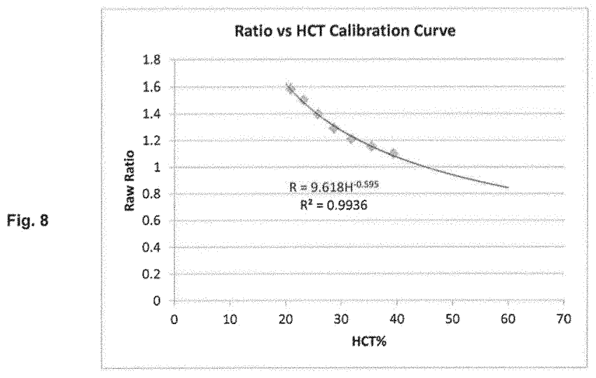

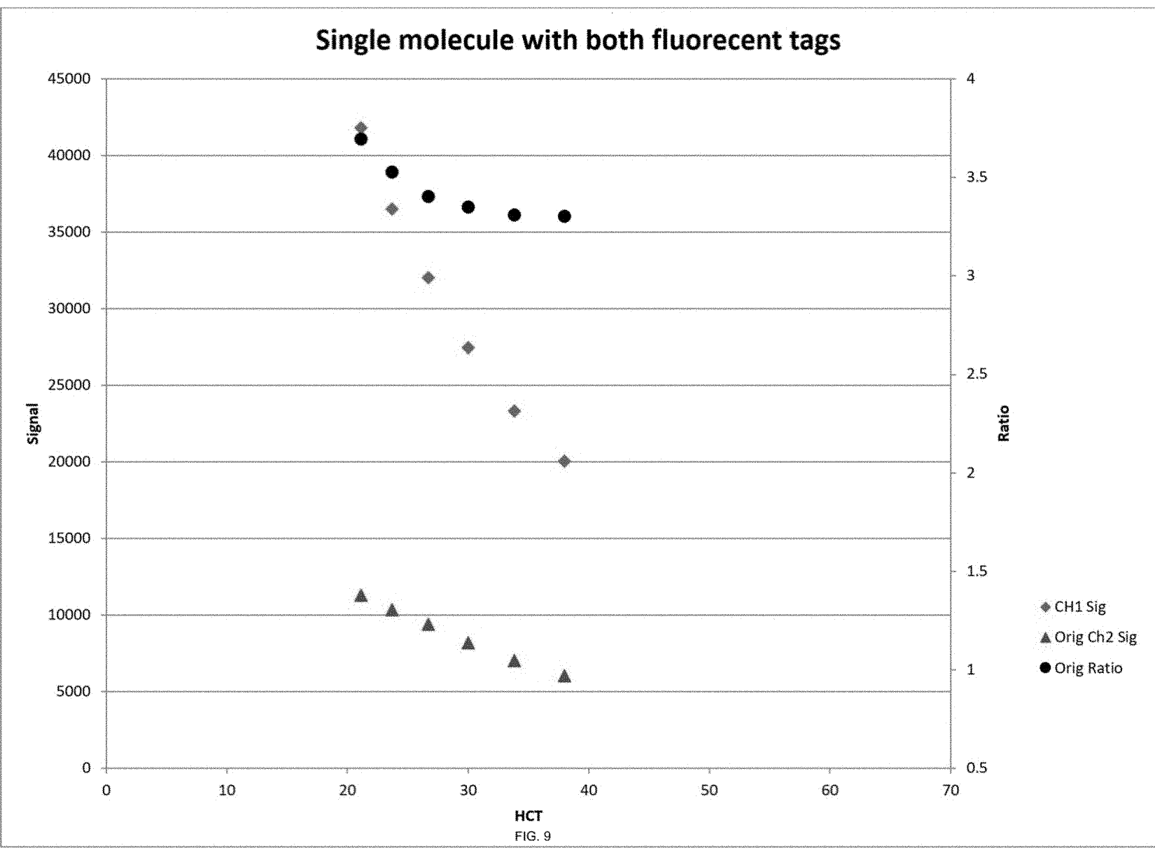

Calibration curves used in this example are shown in FIGS. 6 to 8. FIG. 9 is a calibration curve using one single static marker having two fluorescent tags.

For this example the following set of known parameters is used:

VFI dose concentration: 35 mg/mL of Component 1 and 15 mg/mL of Component 2

Dose Volume: 3.0 mL

To Raw ratio: 1.2

Avg Stable Component 2 Signal Level: 12000

Calibration curve's test volume: 100 mL

Calibration curve's test HCT: 38%.

1. From the raw ratio at T₀, R_{T0}, the apparent HCT of the subject is calculated from the Ratio vs HCT Calibration Curve.

1.2=9.618H^{-0.595} (23)

Hₐₚₚ=33% (24)

2. Using the calculated apparent HCT and the Signal Level vs HCT Calibration Curve; the amount of correction, C, needed to be applied to the average signal level of component 2 (S_{avg}), is calculated using the following (25, 26, 27).

S_{calib}=31200(38)^{-0.3} (25)

S_{calib}=10476

Sₐₚₚ=31200(33)^{-0.3} (26)

Sₐₚₚ=10930

Hₐₚₚ<H_{calib} so S_{calib}/Sₐₚₚ:

10476/10930=0.958=C (27)

3. The correction factor, C, is applied to the average signal level of component 2, S_{avg}, from the test data.

(0.958)(12000)=S_{C}

S_{C}=11496 (28)

4. The corrected signal, S_{c}, in equation (16) is used to determine the equivalent amount of material of component 2 based on the Signal Level vs Material Amount Calibration Curve.

11496=5478.2x (29)

x=2.09 mg=x_{eq} (30)

5. From a ratio of the known amount (mg) of VFI component 2 dosed in the subject, X_{sub}, and a known volume used in calibration, V_{distcalib}, to a calculated equivalent amount of component 2 (mg), x_{eq}, and the volume of distribution of the subject, V_{distcalib}, the subject's volume of distribution is calculated.

V_{distcalib}=Vₜ−VₜH_{calib} (31)

V_{distcalib}=100−100*(0.38) (32)

2.07/62=(3*15)/V_{distsub}

V_{distsub}=1334.9 mL (33)

6. The subject's HCT is calculated from the apparent HCT and the HCT offset.

H_{sub}⁼33+5=38% (34)

7. Blood volume is calculated from the volume of distribution of the subject and the calculated subject HCT.

BV=1334.9/0.38

BV=3513 mL (35)

### Example 5: Determining GFR In A Multi-Dose Context

The disclosed formula for multi-dose calculation addresses the plasma volume of any markers taken pre-dose (Blank). With such method, the total concentration of markers in the blood plasma is always calculated and used, since early and late decay rates are handled differently. With such technique, the case of a first test is treated the same as a follow on test, but setting the pre-dose blank values to zero.

The multi-dose formula and assumptions are as follows:

Blank=plasma concentration of any markers taken pre-dose
C1=initial concentration of marker in plasma vs time
C2=Concentration measured just before new follow on dose is given
C3=Concentration vs time after follow on dose is given
A₁=Initial magnitude of the fast decay rate
B₁=Initial magnitude of the slow decay rate
α=fast decay rate
β=slow decay rate
t₁=time since initial dose of marker
t₂=time since initial dose that follow on blank is drawn
t₃=time since follow on dose is given.
mGFR=Calculated Glomerular Filtration Rate
Dose 1=the initial dose
Dose 2=the follow on dose
PV=Measured Plasma Volume at second dose

Assumes Blank=0 which is the concentration measured pre-dose

C1=A₁^{-αt}^{₁}+B₁^{-βt}^{₁} (36)

New Blank taken just before follow on dose at time=t₂

Assumes Blank is a measured value=C2=A₁^{-αt}^{₂}+B₁^{-βt}^{₂} (37)

New clearance=C3=A₂^{-αt}^{₃}+B₂^{-βt}^{₃} (38)

### Therefore:

$$mGFR = \frac{Dose 2}{\frac{{A}_{2}}{α} + \frac{{B}_{2} - C 2}{β}} \qquad ( 39 )$$

Where A₂ B₂ α and β is the new clearance rate measured after the second dose. The symbols α and β are defined above. A₂ and B₂ the initial magnitudes of the fast and slow decay rates of the markers in the second dose. This same equation can be used in any number of follow on doses.

Utilizing the equations and techniques set forth above, a method for determining a biometric indicator such as plasma concentration in a multi-dose context may be practiced.

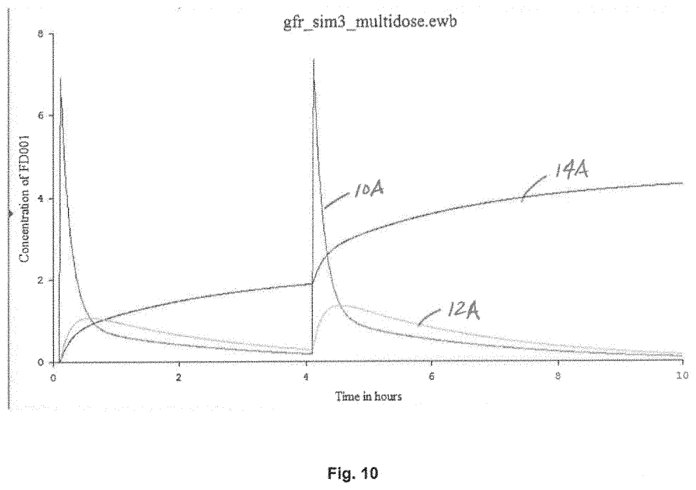

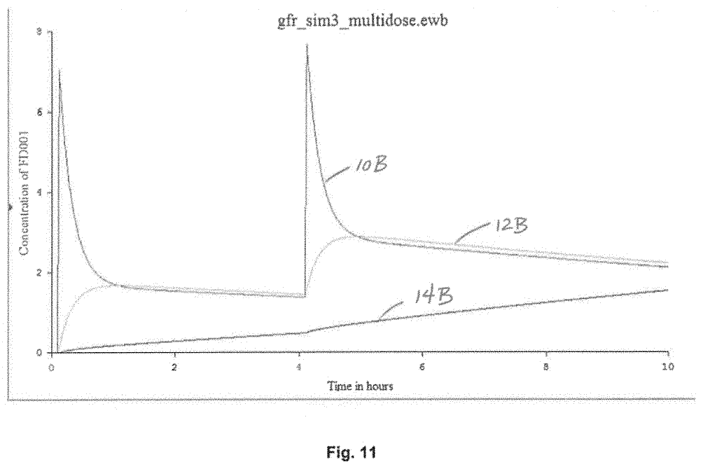

The graphs illustrated in FIGS. 10-11 were generated from a computer simulation model and show how the fast and slow decay curves react during the follow on dose for both a normal and impaired patient. FIG. 10 illustrates a normal mGFR showing a follow on dose of FD001. In FIG. 10, signal 10A (Red) represents the plasma clearance of FD001 in units of ug/ml, while signal 12A (Green) represents the interstitial concentration of the FD001 in units of ug/ml, and signal 14A (Blue) represents the cumulative marker contained in the bladder during the testing time.

FIG. 11 illustrates an impaired mGFR showing a follow on dose of FD001. In FIG. 11, signal 10B (Red) represents plasma clearance of FD001 in units of ug/ml, while signal 12B (Green) represents the interstitial concentration of the FD001 in units of ug/ml, and signal 14B (Blue) represents the cumulative marker contained in the bladder during the testing time. Note that the total kidney clearance remains proportional to total concentration of FD001, while the interstitial leakage is always relative to the new dose.

### Example 6: Calculation of Interstitial Volume

The disclosed formula for calculation of interstitial volume is:

$$\begin{matrix}GFR = \frac{{V}_{1} ( {A}_{1} + {B}_{1} )}{{A}_{1} / α + {B}_{1} / β} & ( 10 ) \\ {V}_{d} = \frac{{V}_{1} ( {A}_{1} + {B}_{1} ) ( \frac{{A}_{1}}{{α}^{2}} + \frac{{B}_{1}}{{β}^{2}} )}{{( \frac{{A}_{1}}{α} + \frac{{B}_{1}}{β} )}^{2}} & ( 11 )\end{matrix}$$

where equation (10) represents GFR from intensity of a single, freely filterable reporter molecule type, and equation (11) represents the volume distribution associated with a single, freely filterable reporter molecule type.

Constants A₂, B₂, α, and β can be obtained by fitting the experiment data to the above equation. Thus, the clearance GFR and the total volume of distribution can be expressed as:

$$\begin{matrix}GFR = \frac{{V}_{1} ( {A}_{2} + {B}_{2} )}{{A}_{2} / α + {B}_{2} / β} & ( 13 ) \\ {V}_{d} = \frac{{V}_{1} ( {A}_{2} + {B}_{2} ) ( \frac{{A}_{2}}{{α}^{2}} + \frac{{B}_{2}}{{β}^{2}} )}{{( \frac{{A}_{2}}{α} + \frac{{B}_{2}}{β} )}^{2}} & ( 14 )\end{matrix}$$

Interstitial Volume is: Vd−PV, wherein PV was calculated by evaluating the concentration change of the dosed marker (e.g., the dosed FD003 marker).

The above-referenced equations work because of the use of a T0 time point concentration in a curve fit computation, as has been set forth herein. If one were to attempt to curve-fit without T0, the above equations would not actually work. The boundary condition of A+B=concentration of the reporter marker at time zero therefore must be true.

### Example 7: Measuring Interstitial Volume in the Case of a Two Exponent Fitting Algorithm

Equations used for calculation of interstitial volume using the derived T0 time point concentration for curve fitting to a two exponent fit employed the following variables:

α=slope of steep line

β=slope of middle line

γ=slope of shallowest line

V₁=the vascular space, or plasma volume (PV) in mL

V₂=the interstitial space in mL

D=the dose of clearance or plasma marker given in μg

t=time

A=magnitude of steep curve

B=magnitude of middle curve

C=magnitude of slowest curve

C₀=the concentration at t=0, or just after the initial dose in μg/mL

C₁=the average concentration over time (t) in μg/mL

G=raw glomerular filtration rate (GFR) in mL/min

λ=measured leakage rate of the marker into V₂ in mL/min

To measure interstitial volume using a two exponent fitting algorithm, the following process was employed, further noting that a small molecule of about 650 Daltons was used for experimental purposes:
1. Data were fitted to a bi-exponential curve fit algorithm with an included time point called Time zero. The known plasma volume was calculated by dividing the dosed concentration of the large dextran marker (150 kD) by the measured average concentration taken from blood samples over the course of the test discussed below. The Dose is 12000 μg of FD003.

$${V}_{1} = \frac{D}{{C}_{1}}$$

$${V}_{1} = \frac{D}{{C}_{1}} = \frac{12000}{4.50} = 2666 mL$$

2. Using the calculated PV (V₁), the concentration of the small or clearance marker at time zero (C₀) could be calculated as:

$${C}_{0} = \frac{323500}{2666} = 121.34 ug \text{/} mL$$

3. The sample data representing the concentrations of the small or clearance marker over time to be fitted using a bi-exponential curve fit algorithm are listed in Table 2 below:

TABLE 2

| Time in minutes | Concentration ug/ml |
|---|---|
| 0 | 121 |
| 10 | 29.2 |
| 120 | 7.27 |
| 150 | 5.83 |
| 180 | 5.19 |
| 210 | 4.21 |
| 320 | 2.25 |

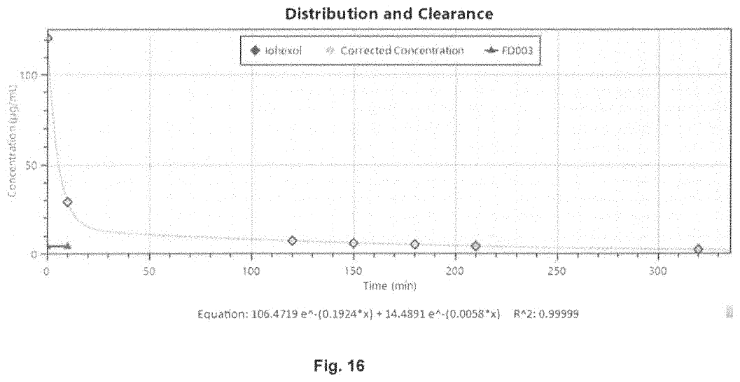

The resulting curve fitted from the above Table 2 data using the bi-exponential curve fit algorithm is illustrated in FIG. 16 and results in the following fitted curve parameters:

A=106.47

α=0.1924

B=14.49

β=0.005847

4. Give the initial dose of cleared marker was D₁=323500 μg/ml, mGFR is then derived by:

$$G = \frac{{D}_{1}}{( \frac{A}{α} + \frac{B}{β} )} = \frac{323500}{\frac{106.47}{0.1924} + \frac{14.49}{.005847}} = 106.7 mL \text{/} min$$

5. Using the computed value of mGFR, the measured leakage rate of the marker into the interstitial space, λ, is then calculated as:

$$λ = \frac{{V}_{1} ( A α + B β )}{A + B} - G = \frac{2666 ( 106.47 * 0.1924 + 14.49 * .005847 )}{106.47 + 14.49} - 106.7 = 348 mL \text{/} min$$

6. Using the computed value of mGFR and λ, the Interstitial Volume is then derived by as:

$${V}_{2} = \frac{λ G}{{V}_{1} αβ} = \frac{348 * 106.7}{2666 * 0.1924 * 0.005847} = 12340 mL$$

### Example 8: Measuring Interstitial Volume in the Case of a Three Exponent Fitting Algorithm

1. Data were fitted to a three-exponential curve fit algorithm with an included time point called Time zero. This point was calculated by dividing the dosed concentration of the small or clearance marker by the known plasma volume. The known plasma volume was calculated by dividing the dosed concentration of the large or plasma marker (150 kD) by the measured average concentration of the large marker taken from blood samples over the course of the test discussed below. The Dose was 12000 μg of FD003. It is further noted that FD001 (of size approximately 7000 Daltons) was also used in the instant Example of fitting.

$${V}_{1} = \frac{D}{{C}_{1}}$$

$${V}_{1} = \frac{D}{{C}_{1}} = \frac{12000}{4.50} = 2666 mL$$

2. Using the calculated PV (V₁), the concentration of the small or clearance marker at time zero (C₀) can be calculated as:

$${C}_{0} = \frac{12000}{2666} = 13.1 μg \text{/} mL$$

3. The sample data representing the concentrations of the small or clearance marker over time to be fitted using a three-exponential curve fit algorithm are listed in Table 3 below:

TABLE 3

| Time in minutes | Concentration ug/ml |
|---|---|
| 0 | 13.1 |
| 15 | 5.00 |
| 30 | 3.15 |
| 60 | 1.80 |
| 120 | 1.04 |
| 170 | 0.654 |
| 310 | 0.486 |

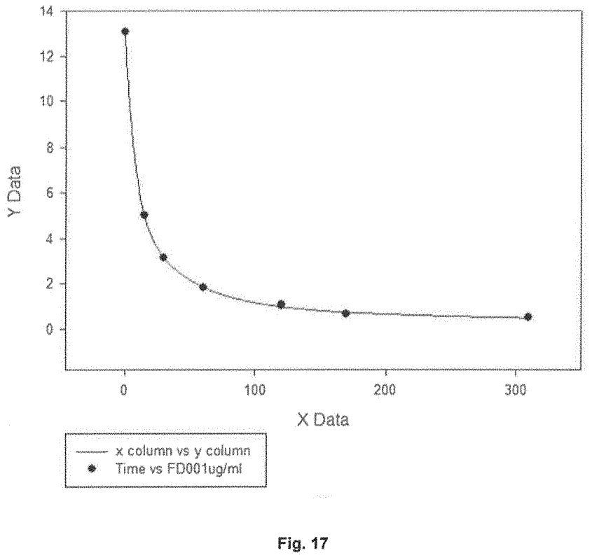

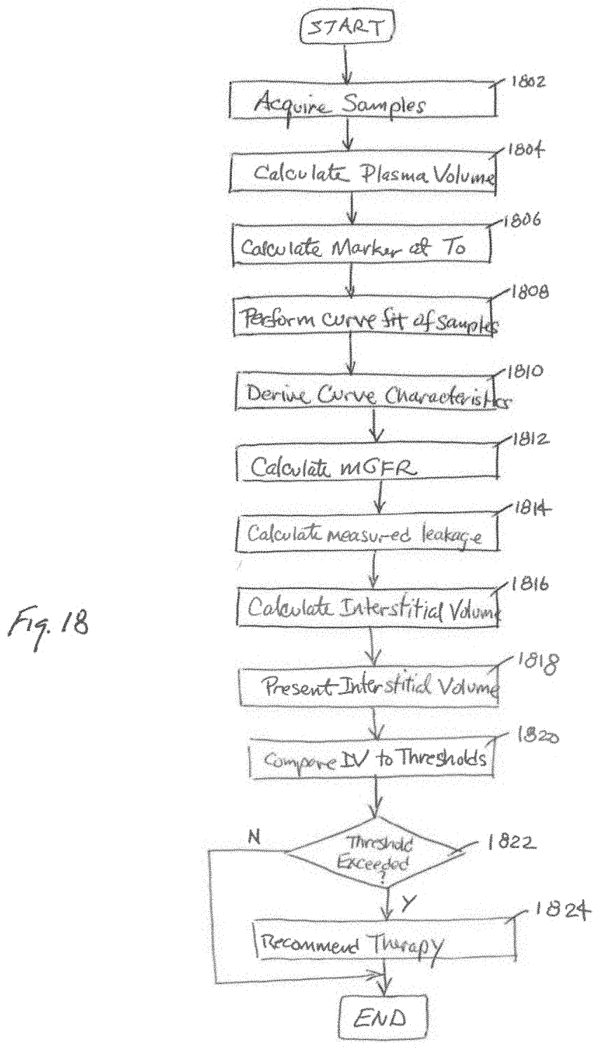

The resulting curve fitted data using a three-exponential curve fit algorithm for the data from the above Table 3 is illustrated in FIG. 17 resulting in:

A=7.6491

α=0.1342

B=4.3982

β=0.0253

C=1.0105

γ=0.0025

4. Give the initial dose of cleared or small marker was D₁=35000 μg/ml, mGFR was then derived by:

$$G = \frac{{D}_{1}}{( \frac{A}{α} + \frac{B}{β} + \frac{C}{γ} )} = \frac{35000}{\frac{106.47}{0.1924} + \frac{14.49}{.005847} + \frac{1.0105}{0.0025}} = 55 mL \text{/} min$$

5. Using the computed value of mGFR, the measured leakage rate of the marker into the interstitial space, λ, is then calculated as:

$$λ = \frac{{V}_{1} ( A α + B β + C γ )}{A + B + C} - G = \frac{2666 ( 7.6491 * 0.1342 + 4.3982 * .0253 + 1.0105 * 0.0025 )}{7.6491 + 14.49 + 1.0105} - 55 = 233.5 mL \text{/} min$$

6. Using the computed value of mGFR and λ, Interstitial Volume was then derived by:

$${V}_{2} = \frac{λ G}{{V}_{1} ( α + β ) γ} = \frac{233.5 * 55.1}{2666 * ( .1342 + .0253 ) * 0.0025} = 12058 mL$$

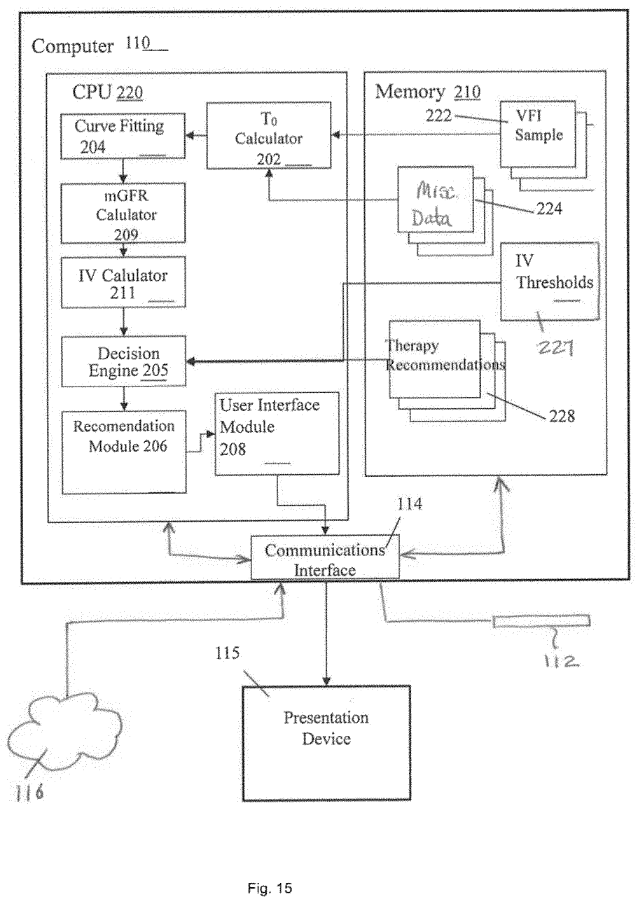

The processes for calculating the interstitial volume as, as described herein, particularly with reference to the above Examples 6-8 herein, is described with reference to the system diagram of FIG. 15 and the flow diagram of FIG. 18. Specifically, to begin, a plurality of VFI sample data 222, representing the concentration of both large and small markers in the blood over the duration of the testing period are acquired, as illustrated by process block 1802. Such acquisition may be done in real-time using peripheral 112 and computer 110 or may be made from previously stored data in memory 210. Next, a value for plasma volume, V₁, is calculated by dividing the dosed concentration of the large marker by the measured average concentration from blood samples, as illustrated by process block 1804. Thereafter, using the calculated plasma volume (V₁), the concentration of the small or clearance marker at time zero (C₀) is calculated, as illustrated by process block 1806. The processes illustrated by blocks 1804 and 1806 may be carried out by a T0 calculation module 202 executable on CPU 220. Next, the data samples are fitted to a curve using the value of C₀ and either a bi-exponential curve fit algorithm or a three-exponential curve fit algorithm by a curve fitting module 204 executing on CPU 220, as illustrated by process block 1808. Next, from the generated curve, values for any of the following fitted curve parameters are derived by a mGFR calculation module 209, as illustrated by process block 1810:

α=slope of steep line

β=slope of middle line

γ=slope of shallowest line

A=magnitude of steep curve

B=magnitude of middle curve

C=magnitude of slowest curve

Next, using the dose of cleared or small marker, D₁, and the calculated fitted curve parameters, a value for the mGFR is then calculated, as illustrated by process block 1812. We note that in step 1810 if a bi-exponential curve fit algorithm is utilized, the value for mGFR can be calculated without values for either the slope of shallowest line, γ, or the magnitude of slowest curve, C. Using the computed value of mGFR, the measured leakage rate of the marker into the interstitial space, λ, is then calculated by the interstitial volume calculator module 211, as illustrated by process block 1814. Using the computed value of mGFR and λ, the Interstitial Volume is then calculated by the interstitial volume calculator module 211, as illustrated by process block 1816. The computed value of the Interstitial Volume and any recommended diagnostic response, along with a graphic representation of the fitted curve and other relevant datum, including values for the initial samples, interim computational values, and patient related information, as illustrated by the sample user via user interface 118, may then be presented to a user via a user interface module 208 executing on CPU 220, as illustrated in process block 1820. The computed value of the Interstitial Volume may be compared by a decision engine module 205 executing on CPU 220 to one or more stored predetermined thresholds 227, or ranges of thresholds, for interstitial volumes associated with certain morphologies or thresholds dynamically generated based on various, characteristics of the subject, such as weight, height, age, etc., as illustrated by decisional step 1820. If a threshold is exceeded, a recommendation engine module 206 executing on CPU 220 may recommend therapy, in the form of a therapeutic and optional dosage thereof, viewable via the user interface 114, to be delivered intravenously to the subject, as illustrated by process blocks 1822 and 1824. The reader will appreciate the relevance of the sample computations and equations set forth in Exhibit 6 and in steps calculation steps 1-6 of Example 7 and 8 relative to the functions of the above described modules of the system 100.

### System Architecture

The techniques and methods disclosed herein may be practiced using system and apparatus as described herein. FIG. 15 is a block diagram illustrating system architecture 100 comprising a computer 110, peripheral device 112, presentation device 115, and network infrastructure 116. Computer 110 comprises a central processing unit (CPU) 200 and memory 210 and communication interface 114. In one embodiment, CPU 200 may be with general purpose processor executing a number of proprietary modules, each of which is programmed to perform specific algorithmic functions, including, but not limited to, T0 calculator module 202, curve fitting module 204, decision engine 205, recommendation engine module 206, user interface module 208, mGFR calculator module 209, and interstitial volume calculator module 211. Memory 210 stores values for VFI sample data 222, calibration and other miscellaneous data values 224, predetermined interstitial volume thresholds 227 and, if an interstitial volume threshold is succeeded within the context of a disease, therapy recommendations 228, as illustrated in FIG. 15 therein. The processes for calculating the interstitial volume via the interaction of the various algorithmic modules executable computer 110, is described in detail with reference to the flow diagram of FIG. 18. Computer 110 further comprises a communications interface 114 which enables the computer 110 to interact with peripheral 112, presentation device 115 and network infrastructure 116.

Embodiments of the above-described systems and methods can be implemented in digital electronic circuitry, in computer hardware, firmware, software and/or combinations thereof. The implementation can be as a computer program product. The implementation can, for example, be in a machine-readable storage device, for execution by, or to control the operation of, data processing apparatus. The implementation can, for example, be a programmable processor, a compute, and/or multiple computers.

A computer program is provided in any form of programming language, including compiled and/or interpreted languages, and the computer program can be deployed in any form, including as a stand-alone program or as a subroutine, element, and/or other unit suitable for use in a computing environment. A computer program can be deployed to be executed on one computer or on multiple computers at one site.

Various mathematical computations and steps of the methods described herein may be performed by one or more programmable processors executing a computer program to perform functions of the disclosed methods by operating on input data and generating output. Method steps can also be performed by apparatus implemented as special purpose logic circuitry. The circuitry can, for example, be a FPGA (field programmable gate array) and/or an ASIC (application-specific integrated circuit). Subroutines and software agents can refer to portions of the computer program, the processor, the special circuitry, software, and/or hardware that implement that functionality.

Components of the system of the present disclosure can be embodied as circuitry, programmable circuitry configured to execute applications such as software, communication apparatus applications, or as a combined system of both circuitry and software configured to be executed on programmable circuitry. Embodiments may include a machine-readable medium storing a set of instructions which cause at least one processor to perform the described methods steps. Machine-readable medium is generally defined as any storage medium which can be accessed by a machine to retrieve content or data. Examples of machine readable media include but are not limited to magneto-optical discs, read only memory (ROM), random access memory (RAM), erasable programmable read only memories (EPROMs), electronically erasable programmable read only memories (EEPROMs), solid state communication apparatuses (SSDs) or any other machine-readable device which is suitable for storing instructions to be executed by a machine such as a computer.

Processors suitable for the execution of a computer program include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer can be operatively coupled to receive data from and/or transfer data to one or more mass storage devices for storing data (e.g., magnetic, magneto-optical disks, or optical disks).

In embodiments, peripheral device 112 may be implemented with an optical probe coupled to the computer 110 through the appropriate optical couplings for sampling of the data and storage into memory 210. In embodiments, probe comprises a “optical conduit” or transparent optical waveguide, such as a fiber optic cable or an optically reflective pipe, which is capable of transmitting optical signals from one location to another. An optical conduit may include an optical waveguide, such as a single fiber optic cable, or multiple optical waveguides arranged about a common optical source and optical interface, such as a bundle of fiber optic cables. In embodiments, the optical conduit has a proximal end and a distal end, with the distal end forming a non-invasive interface between the optical conduit and the vascular system so that the fluorescent intensity of a fluorescent molecule in the vascular system is transmitted from the vascular system to the optical conduit through the optical interface at the distal end of the optical conduit to the proximal end of the optical conduit. The proximal end of the optical conduit may be connected to a fluorescence detector to monitor the fluorescent intensities of the fluorescent markers in the vascular system. The optical conduit may transcend the oral stabilizing guide, or may be set in mechanical communication with the surface of the stabilizing guide such that the oral stabilizing guide limits the movement of the optical conduit. The oral stabilizing guide may include a dental inset. An oral stabilizing guide may also contain an optical guide protrusion for maintaining position of an optical conduit under the tongue.

The oral probe may further include a sterile sheath, which may include a uniform transparent material, or may include a transparent region and a moveable region. The oral probe may further include a fitted region for maintaining a transparent sterile barrier between the optical interface and the tissue portion, or a movable region for maintaining a sterile barrier between the optical positioning guide and the biological environment. Light sources for exciting the fluorescent tags are known in the art. In the case of probes, the light source may be integral with the probe or separate therefrom.

To provide for interaction with a user, the above described techniques can be implemented on a computer having a display device 115. In embodiments, the display device 115 may comprise a dedicated display monitor, such as liquid crystal display (LCD) monitor, or may be implemented with any number of display devices including, but not limited to a computer with a browser device, a telephone, an IP phone, a mobile device (e.g., cellular phone, personal digital assistant (PDA) device, laptop computer, electronic mail device), and/or other communication devices. The browser device includes, for example, a computer (e.g., desktop computer, laptop computer) with a world wide web browser (e.g., Microsoft® Internet Explorer® available from Microsoft Corporation, Mozilla® Firefox available from Mozilla Corporation). The mobile computing device includes, for example, a smartphone or tablet (e.g., iPhone®, iPad®, Android® device, Windows Phone®, etc.).

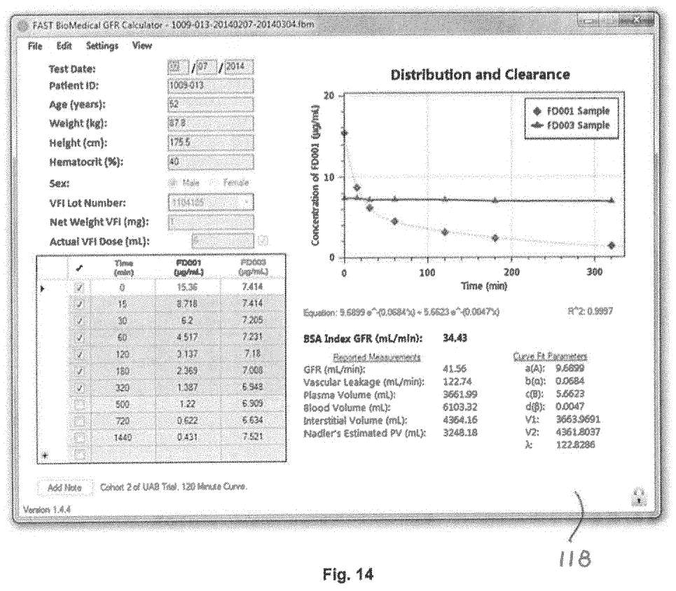

A user interface, such as user interface 118 that illustrated in FIG. 14, is rendered by user interface module 208 of computer 110. Such user interface may include one or more touch sensitive elements which allow the user to select access and manipulate information based on interaction with visual icons rendered on the user interface. In addition, interaction with a user can, for example, be via a keyboard and a pointing device (e.g., a mouse or a trackball) by which the user can provide input to the computer (e.g., interact with a user interface element). Other kinds of devices can be used to provide for interaction with a user. Other devices can, for example, be feedback provided to the user in any form of sensory feedback (e.g., visual feedback, auditory feedback, or tactile feedback). Input from the user can, for example, be received in any form, including acoustic, speech, and/or tactile input.

The above described techniques can be implemented in a distributed computing system that includes a back-end component. The back-end component can, for example, be a data server, a middleware component, and/or an application server. The above described techniques can be implemented in a distributing computing system that includes a front-end component. The front-end component can, for example, be a client computer having a graphical user interface, a Web browser through which a user can interact with an example implementation, and/or other graphical user interfaces for a transmitting device. The components of the system can be interconnected by any form or medium of digital data communication (e.g., a communication network). Examples of communication networks include a local area network (LAN), a wide area network (WAN), the Internet, wired networks, and/or wireless networks which can computer 110 to any other resources or processing elements in a computer network infrastructure illustrated in FIG. 15 as cloud network 116.

Data transmission and instructions can also occur over a communications network. Information carriers suitable for embodying computer program instructions and data include all forms of non-volatile memory, including by way of example semiconductor memory devices. The information carriers can, for example, be EPROM, EEPROM, flash memory devices, magnetic disks, internal hard disks, removable disks, magneto-optical disks, CD-ROM, and/or DVD-ROM disks. The processor and the memory can be supplemented by, and/or incorporated in special purpose logic circuitry.

The various processing steps required to achieve the objectives of the disclosed methods may be delineated into a client/server model in which one or more processes execute as clients and others as servers. In embodiments, the relationship of client and server arises by virtue of computer programs running on the respective computers and having a client-server relationship to each other. In such a system, a client process and a server process are generally remote from each other and typically interact through a communication network infrastructure is the right will see you tomorrow Such networks may include any known network infrastructure components or apology including both packet-switched and circuit-switched networks or any combination thereof. Packet-based networks can include, for example, the Internet, a carrier internet protocol (IP) network (e.g., local area network (LAN), wide area network (WAN), campus area network (CAN), metropolitan area network (MAN), home area network (HAN)), a private IP network, an IP private branch exchange (IPBX), a wireless network (e.g., radio access network (RAN), 802.11 network, 802.16 network, general packet radio service (GPRS) network, HiperLAN), and/or other packet-based networks. Circuit-based networks can include, for example, the public switched telephone network (PSTN), a private branch exchange (PBX), a wireless network (e.g., RAN, bluetooth, code-division multiple access (CDMA) network, time division multiple access (TDMA) network, global system for mobile communications (GSM) network), and/or other circuit-based networks.

The terms comprise, include, and/or plural forms of each are open ended and include the listed parts and can include additional parts that are not listed. The term and/or is open ended and includes one or more of the listed parts and combinations of the listed parts.

The reader will appreciate that the system and techniques disclosed herein enable practitioners to obtain a fairly accurate representation of certain biometric indicators, including volume of the interstitial space which may be useful in diagnosing certain disease conditions including, but limited to any of congestive heart failure, hypertension, chronic kidney disease or sepsis.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments.

What is claimed is:

1. A method of selecting a treatment for a subject at risk of a disease, the method comprising:
A) administering a first VFI to the subject, wherein the first VFI is filtered by the glomeruli of the subject;
B) administering a second VFI to the subject, wherein the second VFI is not filtered by the glomeruli of the subject;
C) measuring a concentration of both the first VFI and the second VFI in the subject, at a timepoint Tm;
D) determining the vascular volume of distribution of both the first VFI and the second VFI at Tm;
E) calculating a T₀ concentration (C_{T0}) for the first VFI by multiplying the concentration of the second VFI concentration at Tm by (first VFI concentration at Tm)/(second VFI concentration at Tm);
F) calculating interstitial volume of the subject from the C_{T0}; and
G) selecting one or more treatments for administration to the subject when a calculated interstitial volume exceeds a threshold value for interstitial volume that would classify subject as in need of a treatment and/or modulation of treatment for a disease.

2. The method of claim 1, further comprising administering the selected treatment to the subject.

3. The method of claim 2 wherein administering the selected treatment to the subject is done by intravenous injection.

4. A method of selecting a treatment for a subject having or at risk of a disease comprises:
A) determining a vascular volume of distribution of both a first VFI and a second VFI at a timepoint Tm from a plurality of sample data values from the subject, the first VFI filterable by the glomeruli of the subject and the second VFI not filterable by the glomeruli of the subject;
B) calculating a T₀ concentration (C_{T0}) value for the first VFI by multiplying a concentration of the second VFI concentration at Tm by a ratio of the first VFI concentration at Tm to the second VFI concentration at Tm;
C) calculating interstitial volume of the subject from the C_{T0} value and measured concentrations; and
D) selecting one or more treatments for administration to the subject when a calculated interstitial volume exceeds a threshold value for interstitial volume that would classify subject as in need of a treatment and/or modulation of treatment.

5. The method of claim 4, further comprising:
E) obtaining data representing a measured concentration of both a first VFI and a second VFI in a subject, at a timepoint Tm.

6. The method of claim 4, further comprising:
E) administering the selected treatment to the subject.

7. A system for calculating the interstitial volume of a patient comprises:
A) a peripheral device operational to measure a concentration of both a first VFI and a second VFI in a subject, at a timepoint Tm, the first VFI filterable by the glomeruli of the subject and the second VFI not filterable by the glomeruli of the subject;
B) a memory operational to store a plurality of measured concentration values of the first VF I and second VFI, a plurality of threshold values of interstitial volume and a plurality of treatment recommendations associated with the threshold interstitial volume values;
C) a processor, coupled to the peripheral device and memory and operational to:
i) determine a vascular volume of distribution of both the first VFI and the second VFI at Tm;
ii) calculate a T₀ concentration (C_{T0}) for the first VFI by multiplying the concentration of the second VFI concentration at Tm by the ratio of (first VFI concentration at Tm)/(second VFI concentration at Tm),
iii) calculate interstitial volume of the subject from the C_{T0} value and measured concentrations at Tm; and
D) a presentation device operatively coupled to the processor and the memory and operational to present the calculated interstitial volume.

8. The system of claim 7, wherein the presentation device is a display device and is further operational to present a user interface enabling selection of one or more treatments for administration to the subject, when the calculated interstitial volume exceeds a threshold value for interstitial volume that would classify subject as in need of a treatment and/or modulation of treatment.

9. The system of claim 7 the presentation device comprises one of a visual display and an audio transducer to present any of a calculated interstitial volume, an exceeded threshold value, or a recommended selected treatment to a user.

10. The system of claim 8, wherein the presentation device comprises a visual display device.

11. The system of claim 7, wherein the peripheral device comprises a probe.

12. The system of claim 11, wherein the peripheral device comprises an oral probe disposable sublingually within an oral cavity of the subject, the oral probe comprising an optical conduit couplable to the processor.

13. The system of claim 7, wherein the processor comprises spectrometric analyzer.

14. A computer program product for use with a computer system operatively coupled to a peripheral, the computer program product comprising a non-transitory medium having computer readable instructions embedded thereon comprising:
A) program code for measuring a concentration of both a first VFI and a second VFI in a subject, at a timepoint Tm, the first VFI filterable by the glomeruli of the subject and the second VFI not filterable by the glomeruli of the subject;
B) program code for determining a vascular volume of distribution of both the first VFI and the second VFI at Tm;
C) program code for calculating a T₀ concentration (C_{T0}) for the first VFI by multiplying the concentration of the second VFI concentration at Tm by the ratio of (first VFI concentration at Tm)/(second VFI concentration at Tm), or by a proxy for such comparison of the first VFI concentration at Tm to the second VFI concentration at Tm;
D) program code for calculating interstitial volume of the subject from the C_{T0} value and measured concentrations at Tm; and
E) program code for causing the calculated interstitial volume to be presented on a device.

15. The computer program product of claim 14 further comprising:
F) program code for generating a user interface enabling selection of one or more treatments for administration to the subject, when the calculated interstitial volume exceeds a threshold value for interstitial volume that would classify subject as in need of a treatment and/or modulation of treatment.

16. The method of claim 1 wherein the disease comprises one of congestive heart failure, hypertension, kidney disease or sepsis.

17. A method of selecting a treatment for a subject having or at risk of a disease based on a value for the interstitial space volume of the subject, the method comprising:
A) acquiring a plurality of sample data values representing concentrations of a small marker and a large marker in blood samples of a subject over a duration of time, the small marker filterable by glomeruli of the subject and the large marker not filterable by the glomeruli of the subject;
B) calculating a value for plasma volume of the subject, V₁, by dividing a dosed concentration of the large marker provided to the subject by a measured average concentration of the large marker from the plurality of sample data values;
C) calculating a value for concentration of the small marker at time zero (C₀) using the calculated vale of the plasma volume (V₁);
D) fitting the plurality of sample data values of at least the small marker to a curve using the value of C₀;
E) calculating a plurality of values for parameters of a resulting fitted curve;
F) calculating a value for mGFR using the plurality of values for parameters of the fitted curve and a value for an initial dose of the small marker provided to a Sam the subject;
G) deriving a value for a measured leakage rate of the small marker into interstitial space of the subject using the calculated value of the mGFR and the calculated plurality of values for parameters of the fitted curve;
H) deriving a value for the interstitial space volume of the subject using the derived value for the measured leakage rate of the small marker into the interstitial space and the calculated value of mGFR and the calculated plurality of values for parameters of the fitted curve; and
I) selecting one or more treatments for administration to the subject when the derived value for the interstitial space volume exceeds a threshold value for interstitial space volume that would classify the subject as in need of a treatment and/or modulation of treatment for a disease.
